(19) United States
(12) Patent Application Publication
(10) Pub. No.: US 2004/0091891 A1
(43) Pub. Date: May 13, 2004

(54) Novel enzymes and genes coding for the same derived from methylophilus methylotrophus

(76) Inventors: Yurgis Antanas Vladovich Iomantas, Moscow (RU); Elena Georgievna Abalakina, Moscow (RU); Yoshihiro Usuda, Kawasaki-shi (JP); Yosuke Nishio, Kawasaki-shi (JP); Natalia Vasilievna Groshkova, Moscow (RU)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/416,021

(22) PCT Filed: Nov. 13, 2001
(86) PCT No.: PCT/JP01/09926

(30) Foreign Application Priority Data
Nov. 13, 2000 (RU) 2000128122

(51) Int. Cl. C12Q 1/68; C07H 21/04; C12P 19/02; C12N 9/02

(52) U.S. Cl. 435/6; 435/69.1; 435/105; 435/189; 435/320.1; 435/325; 536/23.2

(57) ABSTRACT

There are provided novel 3-deoxy-D-arabinoheptulosonate-7-phosphate synthase and prephenate dehydratase/chorismate mutase and DNAs coding the enzymes derived from Methylophilus methylotrophus.

### TECHNICAL FIELD

The present invention relates to biotechnology, and more specifically to 3-deoxy-D-arabinoheptulosonate-7-phosphate synthase, prephenate dehydratase and genes coding for the enzymes. The genes are useful for improvement of productivity of aromatic amino acids.

### BACKGROUND ART

Conventionally, L-amino acids have been industrially produced by fermentation method utilizing microorganisms belonging to the genera Brevibacterium, Corynebacterium, Bacillus, Escherichia, Streptomyces, Pseudomonas, Arthrobactor, Serratia, Penicillum and Candida. As these microorganisms, strains isolated from nature or mutants of these microorganisms have been used to improve the productivity. Further, there have been disclosed various recombinant DNA techniques to improve L-amino acids productivity by enhancing enzymatic activities involving in L-amino acid-biosynthetic pathways.

Though the productivity of L-amino acids has been improved by breeding of aforementioned microorganisms or improving production processes, it is still desired to develop more inexpensive and efficient processes for producing L-amino acids in order to meet the expected markedly increased future demand of the L-amino acids.

Conventionally, there have been known the methods for producing amino acids by fermentation using methanol as raw material which is able to get inexpensively and massively, utilizing the bacteria belonging to genera Achromobactor and Pseudomonas (See Japanese Patent Laid-open No. 45-25273), Protaminobactor (See Japanese Patent Laid-open No. 49-125590), Protaminobactor and Methanomonas (See Japanese Patent Laid-open No. 5025790), Microcyclus (See Japanese Patent Laid-open No. 52-18886), Methylobacillus (See Japanese Patent Laid-open No. 4-91793) and Bacillus (See Japanese Patent Laid-open No. 3-505284).

Besides, there are several enzymes that play a central role in the biosynthetic pathway of aromatic compounds such as L-phenylalanine, L-tyrosine and L-tryptophan. The key enzyme is 3-deoxy-D-arabinoheptulosonate-7-phosphate synthase (hereafter abbreviated as “DS”). For biosynthesis of L-phenylalanine, prephenate dehydratase (hereafter abbreviated as “PD”) is also key enzyme.

However, it has not been known either gene encoding DS and PD of a bacterium belonging to the genus Methylophilus.

### Disclosure Of The Invention

An object of the present invention is to provide the genes encoding DS and PD of a bacterium belonging to the genus Methylotrophus.

To achieve the aforementioned object, the present inventors intensively studied. As a result, they succeeded in isolating genes coding for DS and PD from Methylophilus methylotrophus using chorismate mutase-prephenate dehydratase gene (pheA)-deficient strain of Escherichia coli, and have completed the present invention.

That is, the present invention provides:

(1) A protein as defined in the following (A) or (B):

(A) a protein which comprises the amino acid sequence depicted in SEQ ID NO: 2; or

(B) a protein which comprises the amino acid sequence including deletion, substitution, insertion or addition of one or several amino acids in the amino acid sequence depicted in SEQ ID NO: 2 and which has the 3-deoxy-D-arabinoheptulosonate-7-phosphate synthase activity.

(2) A DNA coding for a protein as defined in the following (A) or (B):

(A) a protein which comprises the amino acid sequence depicted in SEQ ID NO: 2; or

(B) a protein which comprises the amino acid sequence including deletion, substitution, insertion or addition of one or several amino acids in the amino acid sequence depicted in SEQ ID NO: 2 and which has the 3-deoxy-D-arabinoheptulosonate-7-phosphate synthase activity.

(3) The DNA according to (2), which is a DNA as defined in the following (A) or (B):

(A) a DNA which comprises the nucleotide sequence depicted in SEQ ID NO: 1; or

(B) a DNA which is hybridizable with the nucleotide sequence depicted in SEQ ID NO: 1 or the probe prepared from said sequence under stringent condition and which code for a protein which has 3-deoxy-D-arabinoheptulosonate-7-phosphate synthase activity.

(4) The DNA according to (3), wherein the stringent condition is the condition in which washing is performed at 60° C., and at a salt concentration corresponding to 1×SSC and 0.1% SDS.

(5) A protein as defined in the following (C) or (D):

(C) A protein which comprises the amino acid sequence depicted in SEQ ID NO: 4; or

(D) A protein which comprises the amino acid sequence including deletion, substitution, insertion or addition of one or several amino acids in the amino acid sequence depicted in SEQ ID NO: 4 and which has at least one of the prephenate dehydratase activity or the chorismate mutase activity.

(6) A DNA coding for a protein as defined in the following (C) or (D):

(C) A protein which comprises the amino acid sequence depicted in SEQ ID NO: 4; or

(D) A protein which comprises the amino acid sequence including deletion, substitution, insertion or addition of one or several amino acids in the amino acid sequence depicted in SEQ ID NO: 4 and which has at least one of the prephenate dehydratase or the chorismate mutase activity.

(7) The DNA according to (6), which is a DNA as defined in the following (c) or (d):

(c) A DNA which comprises the nucleotide sequence depicted in SEQ ID NO: 3; or

(d) A DNA which is hybridizable with the nucleotide sequence depicted in SEQ ID NO: 3 or the probe prepared from said sequence under stringent condition and which code for a protein which has at least one of the prephenate dehydratase or chorismate mutase activity.

(8) The DNA according to (3), wherein the stringent condition is the condition in which washing is performed at 60° C., and at a salt concentration corresponding to 1×SSC and 0.1% SDS.

In the present invention, the term “3-deoxy-D-arabinoheptulosonate-7-phosphate synthase activity” means an activity which catalyses a reaction to synthesize 3-deoxy-D-arabinoh ptulosonate-7-phosphate from phosphoenolpyruvate and D-erythrose 4-phosphate. The term “prephenate dehydratase activity” means an activity which catalyses a reaction to synthesize phenylpyruvic acid from prephenic acid, the term “chorismate mutase” means an activity which catalyses a reaction to synthesize prephenic acid from chorismic acid. It is suggested that PD described in the present invention has chorismate mutase activity as well as prephenate dehydratase activity like other microorganism such as Escherichia coli. In the present invention, the term “at least one of prephenate dehydratase or chorismate mutase activity” means one or both of the properties which PD possesses. Hereafter, in the present invention, “at least one of prephenate dehydratase or chorismate mutase activity” may be referred to as “PD activity”.

The present invention will be explained in detail hereinafter.

The DNAs of the present invention may be obtained from chromosomal DNA of M. methylotrophus as described below.

Chromosomal DNA of M. methylotrophus, for example, M. methylotrophus strain AS-1 is prepared. Chromosomal DNA can be obtained from the cell pellet by means of, for example, a method of Saito and Miura (Biochem. Biophys. Acta., 72, 619 (1963)), or a method of K. S. Kirby (Biochem. J., 64, 405 (1956)).

Then, in order to isolate the DS or PD gene from the chromosomal DNA thus obtained, a chromosomal DNA library is prepared. At first, the chromosomal DNA is partially digested with a suitable restriction enzyme to obtain a mixture of various fragments. A wide variety of restriction enzymes can be used if the degree of cutting is controlled by the cutting reaction time and the like. For example, Sau3AI or BamHI is allowed to react on the chromosomal DNA at a temperature not less than 30° C., preferably at 37° C. at an enzyme concentration of 1-10 units/ml for various periods of time (1 minute to 2 hours) to digest it.

Next, obtained DNA fragments are ligated with a vector DNA autonomously replicable in cells of bacteria belonging to the genus Escherichia to prepare recombinant DNA. Concretely, a restriction enzyme, which generates the terminal nucleotide sequence complement to that generated by the restriction enzyme Sau3AI used to cut the chromosomal DNA, for example, BamHI, is allowed to act on the vector DNA under a condition of a temperature not less than 30° C. and an enzyme concentration of 1-100 units/ml for not less than 1 hour, preferably for 1-3 hours to completely digest it, and cut and cleave it. Next, the chromosomal DNA fragment mixture obtained as described above is mixed with the cleaved and cut vector DNA, on which DNA ligase, preferably T4 DNA ligase is allow d to act under a condition of a temperature of 4-16° C. at an enzyme concentration of 1-100 units/ml for not less than 1 hour, preferably for 4-24 hours to obtain recombinant DNA.

The obtained recombinant DNA is used to transform a microorganism belonging to the genus Escherichia, for example, such as Escherichia coli B-7078 (pheA::Tn10(Km^{R})). Then the transformants are plated on agar plates without phenylalanine and resulted colonies are inoculated in a liquid medium and cultivated. Plasmids are recovered from the cells to obtain DNA fragment containing PD gene.

Whether the DNA fragment obtained as described above actually contains PD gene or not is confirmed by sequencing the fragment and by confirming the determined sequence contains the sequence depicted in SEQ ID NO: 3.

It was proved that the cloned fragment containing PD gene obtained in Example described later also contains DS gene by the fact that the fragment complements aromatic auxotrophy of AB3257 strain (aroG365⁻, aroH367⁻, aroF363⁻, thi-1, ilvC7, argE3, his-4, proA2, xyl-5 galK2, lacY1, mtl-1, strA712, tfr3, tsx-358, supE44, hsdR2, zjj-202::Tn10) which is a DS-deficient strain, and by sequencing of the fragment.

In case BamHI is used to digest chromosomal DNA of Methylophilus methylotrophus AS-1 strain, the DS and PD gene is cloned as about 10 Kb BamHI-fragment.

The DS and PD gene of other bacterium belonging to genus Methylotrophus can be isolated by the same manner as described above. Further, since nucleotide sequence of the DNA of the present invention is clarified, the DNA can be obtained from chromosomal DNA or genomic library of a bacterium belonging to genus Methylophilus by PCR (polymerase chain reaction) utilizing oligonucleotides synthesized based on the determined sequence as a primer or hybridization utilizing oligonucleotide as described above as a probe.

It may be used normal methods which are well known to the person skilled in the art to perform preparation of genomic DNA, preparation of genomic DNA library, hybridization, PCR, preparation of plasmid DNA, digestion and ligation of DNA, and transformation. These are described by Sambrook, J., Fritsch, E. F., and Maniatis, T., “Molecular Cloning A Laboratory Manual, Second Edition”, Cold Spring Harbor Laboratory Press, (1989).

A nucleotide sequence of DS gene obtained as described above is illustrated in SEQ ID NO: 1 in Sequence Listing. Further, an amino acid sequence of a protein which may be encoded by nucleotide sequence is illustrated in SEQ ID NO: 2.

A nucleotide sequence of PD gene obtained as described above is illustrated in SEQ ID NO: 3 in Sequence Listing. Further, an amino acid sequence of a protein which may be encoded by the nucleotide sequence is illustrated in SEQ ID NO: 4.

The DNA of the present invention may code for DS or PD including substitution, deletion, insertion, addition, or inversion of one or several amino acids at one or a plurality of positions, provided that the activity of DS or PD encoded thereby is not deteriorated. The number of “several” amino acids differs depending on the position or the type of amino acid residues in the three-dimensional structure of the protein. This is because of the following reason. That is, some amino acids such as isoleucine and valine are amino acids having high homology to one another. The difference in such an amino acid does not greatly affect the three-dimensional structure of the protein. Therefore, the protein encoded by the DNA of the present invention may be one which has homology of not less than 35 to 50%, preferably 50 to 70% with respect to the entire amino acid residues for constituting DS or PD, and which has the DS and PD activity. More appropriately, the number of “several” amino acids is 2 to 30, preferably 2 to 20, and more preferably 2 to 10.

DNA, which codes for the substantially same protein as DS or PD as described above, is obtained, for example, by modifying the nucleotide sequence, for example, by means of the site-directed mutagenesis method so that one or more amino acid residues at a specified site involve substitution, deletion, insertion, addition, or inversion. DNA modified as described above may be obtained by the conventionally known mutation treatment. The mutation treatment includes a method for treating DNA coding for DS and PD in vitro, for example, with hydroxylamine, and a method for treating a microorganism, for example, a bacterium belonging to the genus Escherichia harboring DNA coding for DS and PD with ultraviolet irradiation or a mutating agent such as N-methyl-N′-nitro-N-nitrosoguanidine (NTG) and nitrous acid usually used for the mutation treatment.

The substitution, deletion, insertion, addition, or inversion of nucleotide as described above also includes mutation (mutant or variant) which naturally occurs, for example, on the basis of the individual difference or the difference in species or genus of the microorganism harboring DS and PD.

The DNA, which codes for the substantially same protein as DS and PD, is obtained by expressing DNA having mutation as described above in an appropriate cell, and investigating the DS and PD activity of an expressed product. The DNA, which codes for the substantially same protein as DS and PD, is also obtained by isolating DNA that is hybridizable with DNA having, for example, a nucleotide sequence depicted in SEQ ID NO: 1 in Sequence Listing under a stringent condition, and which codes for a protein having the DS and PD activity, from DNA coding for DS and PD having mutation or from a cell harboring it. The “stringent condition” referred to herein is a condition under which so-called specific hybrid is formed, and non-specific hybrid is not formed. It is difficult to clearly express this condition by using any numerical value. However, for example, the stringent condition includes a condition under which DNA's having high homology, for example, DNA's having homology of not less than 50% are hybridized with each other, and DNA's having homology lower than the above are not hybridized with each other. Alternatively, the stringent condition is exemplified by a condition under which DNA's are hybridized with each other at a salt concentration corresponding to an ordinary condition of washing in Southern hybridization, i.e., 60° C., 1×SSC, 0.1% SDS, preferably 0.1×SSC, 0.1% SDS.

The gene, which is hybridizable under the condition as described above, includes those having a stop codon generated in a coding region of the gene, and those having no activity due to mutation of active center. However, such mutants can be easily removed by ligating the gene with a commercially available activity expression vector, and measuring the DS and PD activity in accordance with the method as described above.

The host to be expressed DS or PD gene are exemplified by, for example, bacterium belonging to the genus Escherichia such as Escherichia coli, Cornyneform bacterium such as Brevibacterium lactofermentum, bacterium belonging to the genus Methylophilus such as Methylophilus methylotrophus, other various eukaryotes such as Saccharomyces cerevisiae, animal cells and plant cells, preferably prokaryote, especially E. coli Coryneform bacterium and M. methylotrophus.

The vector to be introduced DS or PD gene into E. coli includes, for example, pUC19, pUC18, pBR322, pHSG299, pHSG399, pHSG398, RSF1010, pMW119, pMW118, pMW219 and pMW218. Phage DNA vectors may be also used.

The vector to be to be used for introducing DS or PD gene into Coryneform bacterium includes, for example, pAM330 (see Japanese Patent Laid-open No. 58-67699), pHM1519 (see Japanese Patent Laid-open No. 58-77895), pAJ655, pAJ611 and pAJ1844 (see Japanese Patent Laid-open No. 58-192900), pCG1 (see Japanese Patent Laid-open No. 57-134500), pCG2 (see Japanese Patent Laid-open No. 58-35197), pCG4 and pCG11 (see Japanese Patent Laid-open No. 57-183799), pHK4 (see Japanese Patent Laid-open No. 5-7491).

Introduction of DS and PD gene may be performed by transforming the host as described above with a recombinant vector obtained by connecting DS or PD gene to the vector as described above. The DS or PD gene may be incorporated into the genome of the host in accordance with the method based on the use of transduction, transposon (Berg, D. E. and Berg, C. M., Bio/Technol., 1, 417 (1983)), Mu phage (Japanese Laid-Open Patent Publication No. 2-109985), or homologous recombination (Experiments in Molecular Genetics, Cold Spring Harbor Lab. (1972)).

DS and PD may be produced by cultivating the cell in which DS or PD gene is introduced in accordance with the method as described above, producing and accumulating DS or PD in the medium, collecting from the culture. The medium used for cultivation may be selected appropriately to the host used therein.

DS or PD produced by the method as described above, if necessary, it may be purified from cell extract or medium by normal method of purification of enzymes such as ion exchange chromatography, gel filtration chromatography, absorption chromatography, solvent precipitation and the like.

Microorganism having higher activity of DS or PD than that of wild type may be constructed by using the DNA of the present invention. It can be performed by transforming microorganism with the vector containing DS or PD gene as an expressible form.

Bacterium to be used for the present invention includes, for example, Methylophilus methylotrophus AS1 (NCIMB10515) or the like. It is possible to obtain Methylophilus methylotrophus AS1 (NCIMB10515) from National Collections of Industrial and Marin Bacteria, NCIMB Lts., Torry Res arch Station 135, Abbey Road, Aberdeen AB9 8DG, United Kingdom.

In order to improve the productivity of aromatic amino acids, especially for L-phenylalanine, it is useful to amplification of genes encoding the DS and PD enzyme.

### BRIEF DESCRIPTION OF THE DRAWINGS

FIG. 1 shows the construction of plasmids pPD1 and pPD2 having DS and PD genes, and

FIG. 2 shows the complementation analysis of the plasmids, obtained after the deletion of M. methylotrophus DNA fragment, carrying PD and DS genes.

### BEST MODE FOR CARRYING OUT THE INVENTION

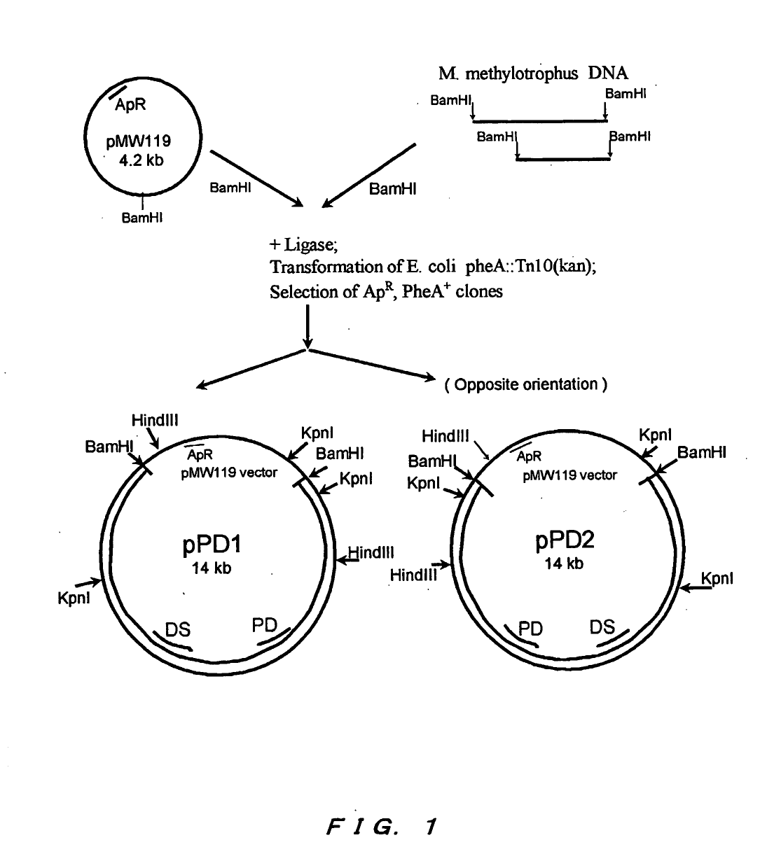

The 10 kbp BamHI DNA fragment carrying the M. methylotrophus prephenate dehydratase and DAHP-synthase genes, was cloned on a low copy vector pMW119 (Ap^{R}) in shotgun experiments by complementation (FIG. 1). In this experiment the chromosomal DNA of the M. methylotrophus AS-1 was digested with BamHI and the resulting DNA fragments were ligated with BamHI digestion product of plasmid pMW119 using T4 DNA ligase. The ligation product was used to transform E. coli B-7078 strain (pheA::Tn10(Km^{R})). Among the clones resistant to ampicillin, the strains in which phenylalanine auxotrophy disappeared were selected, and the recombinant plasmids were recovered from the selected strains. One of the resulted plasmids was named as pPD1. The plasmid with the opposite orientation of the cloned fragment to that of pPD1 was named as pPD2.

The plasmids pPD1 and pPD2 complemented to the prototrophy not only the pheA⁻ mutation of E. coli B-7078 strain, but also the DS-minus E. coli AB3257 strain (aroG⁻, aroH⁻, aroF⁻). Thus, the both plasmids pPD1 and pPD2 were supposed to bear the genes encoding PD enzyme and DS enzyme in the same cloned DNA fragment.

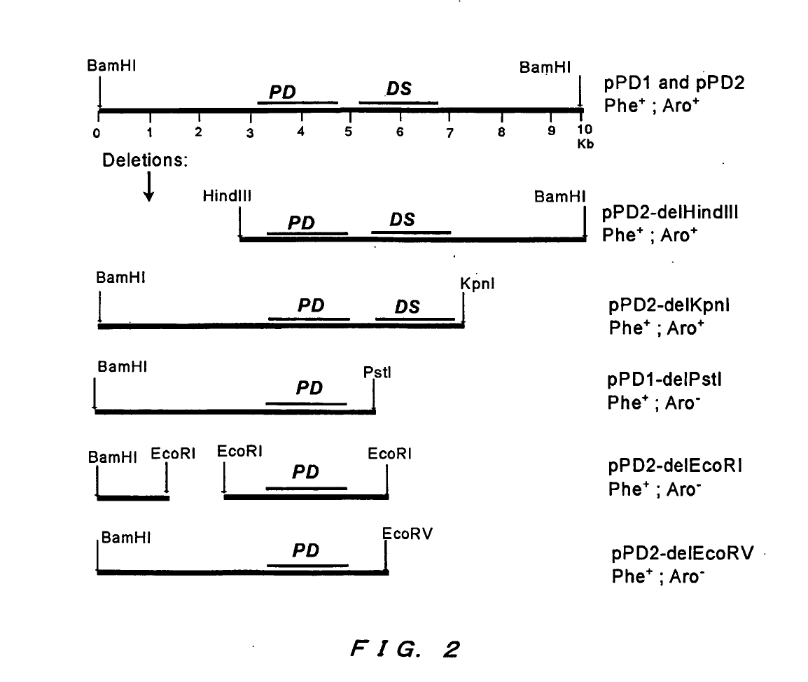

The deletion derivatives of pPD1 and pPD2 were constructed (FIG. 2). The deletions were performed in vitro by digesting the plasmid DNA with different restriction enzymes and ligating resulting DNA fragments. The ligation mixtures were used to transform the PD-minus strain E. coli B-7078 (pheA::Tn10 (kan)) to a Phe⁺ prototrophy. The isolated plasmids were mapped and tested in the ability to complement the DS-minus mutant E. coli AB3257 (aroG⁻, aroH⁻, aroF⁻) to Aro⁺ prototrophy. The constructed deletion derivatives were varied in structure and complementation ability. The deletion derivatives carrying only one gene encoding PD enzyme were found. These deletion derivatives lost an ability to complement DS-minus mutant E. coli AB3257 (aroG⁻, aroH⁻, aroF⁻) to prototrophy. Thus, the cloned M. methylotrophus DNA fragment in pPD1 or pPD2 was supposed to carry two different genes encoding DS and PD enzymes, respectively.

The nucleotide sequences of the M. methylotrophus two genes encoding DS and PD enzymes were determined. The nucleotide sequence of the DS gene and the amino acid sequence coded by the nucleotide sequence are shown in SEQ ID NO: 1. The nucleotide sequence of the PD gene and the amino acid sequence coded by the nucleotide sequence are shown in SEQ ID NO: 3.

The nucleotide sequences of M. methylotrophus genes encoding DS and PD were analyzed and characterized by using the computer programs. The DS and PD gene sequences after translation showed a significant amino acid sequence similarity with the same function proteins of many other microorganisms (Table 1, 2).

### INDUSTRIAL APPLICABILITY

The present invention provides 3-deoxy-D-arabinoheptulosonate-7-phosphate synthase, prephenate dehydratase and genes coding for the enzymes. The genes are useful for improvement of productivity of aromatic amino acids

TABLE 1
The alignment of M. methylotrophus DS enzyme
protein sequence with the similar sequences of other
microorganisms

| | | | | Homology |
|---|---|---|---|---|
| | Sequence | | | (%) with |
| | accession | | | M.m. DS |
| Microorganism | number | Gene | Enzyme | protein |
| Echerichia coli | P00886 | aroG | 3-deoxy-D-arabino- | 60% |
| | | | heptulosonate 7- | |
| | | | phosphate synthase | |
| Haemophilus | P44303 | aroG | 3-deoxy-D-arabino- | 56% |
| influenzae | | | heptulosonate 7- | |
| | | | phosphate synthase | |
| Echerichia coli | P00887 | aroH | 3-deoxy-D-arabino- | 54% |
| | | | heptulosonate 7- | |
| | | | phosphate synthase | |
| Schizosaccha- | Q09755 | aroF | 3-deoxy-D-arabino- | 52% |
| romyces pombe | | | heptulosonate 7- | |
| | | | phosphate synthase | |
| Erwinia herbicola | O54459 | aroH | 3-deoxy-D-arabino- | 52% |
| | | | heptulosonate 7- | |
| | | | phosphate synthase | |
| Saccharomyces | P32449 | aroG | 3-deoxy-D-arabino- | 54% |
| cerevisiae | | | heptulosonate 7- | |
| | | | phosphate synthase | |
| Echerichia coli | P00888 | aroF | 3-deoxy-D-arabino- | 49% |
| | | | heptulosonate 7- | |
| | | | phosphate synthase | |
| Candida albicans | P79023 | aroG | 3-deoxy-D-arabino- | 51% |
| | | | heptulosonate 7- | |
| | | | phosphate synthase | |
| Salmonella | P21307 | aroF | 3-deoxy-D- arabino- | 49% |
| typhimurium | | | heptulosonate 7- | |
| | | | phosphate synthase | |
| Buchnera | P46245 | aroH | 3-deoxy-D-arabino- | 47% |
| aphidicola | | | heptulosonate 7- | |
| | | | phosphate synthase | |
| Saccharomyces | P14843 | aroF | 3-deoxy-D-arabino- | 49% |
| cerevisiae | | | heptulosonate 7- | |
| | | | phosphate synthase | |
| Corynebacterium | P35170 | aroG | 3-deoxy-D-arabino- | 48% |
| glutamicum | | | heptulosonate 7- | |
| | | | phosphate synthase | |
| Candida albicans | P34725 | aroF | 3-deoxy-D- | 50% |
| | | | arabino-heptulosonate | |
| | | | 7-phosphate synthase | |
| Erwinia herbicola | Q02285 | aroF | 3-deoxy-D- | 53% |
| | | | arabino-heptulosonate | |
| | | | 7-phosphate synthase | |
| Amycolatopsis | Q44093 | aroG | 3-deoxy-D- | 52% |
| methanolica | | | arabino-heptulosonate | |
| | | | 7-phosphate synthase | |

TABLE 2
The alignment of M. methylotrophus PD enzyme
protein sequence with the similar sequences of other
microorganisms

| | | | | Homology |
|---|---|---|---|---|
| | Sequence | | | (%) with |
| | accession | | | M.m. PD |
| Microorganism | number | Gene | Enzyme | protein |
| Neisseria | Q9ZHY3 | pheA | Chorismate mutase; | 54% |
| gonorrhoeae | | | Prephenate dehydratase | |
| Pseudomonas | P27603 | pheA | Chorismate mutase; | 47% |
| stutzeri | | | Prephenate dehydratase | |
| Aquifex | O67085 | pheA | Chorismate mutase; | 47% |
| aeolicus | | | Prephenate dehydratase | |
| Erwinia | Q02286 | pheA | Chorismate mutase; | 37% |
| herbicola | | | Prephenate dehydratase | |
| Echerichia coli | P07022 | pheA | Chorismate mutase; | 36% |
| | | | Prephenate dehydratase | |
| Haemophilus | P43900 | pheA | Chorismate mutase; | 34% |
| influenzae | | | Prephenate dehydratase | |

1. A protein as defined in the following (A) or (b):
(A) a protein which comprises the amino acid sequence depicted in SEQ ID NO: 2; or
(B) a protein which comprises the amino acid sequence including deletion, substitution, insertion or addition of one or several amino acids in the amino acid sequence depicted in SEQ ID NO: 2 and which has the 3-deoxy-D-arabinoheptulosonate-7-phosphate synthase activity.

2. A DNA coding for a protein as defined in the following (A) or (B):
(A) a protein which comprises the amino acid sequence depicted in SEQ ID NO: 2; or
(B) a protein which comprises the amino acid sequence including deletion, substitution, insertion or addition of one or several amino acids in the amino acid sequence depicted in SEQ ID NO: 2 and which has the 3-deoxy-D-arabinoheptulosonate-7-phosphate synthase activity.

3. The DNA according to claim 2, which is a DNA as defined in the following (A) or (B):
(A) a DNA which comprises the nucleotide sequence depicted in SEQ ID NO: 1; or
(B) a DNA which is hybridizable with the nucleotide sequence depicted in SEQ ID NO: 1 or the probe prepared from said sequence under stringent condition and which code for a protein which has 3-deoxy-D-arabinoheptulosonate-7-phosphate synthase activity.

4. The DNA according to claim 3, wherein the stringent condition is the condition in which washing is performed at 60° C., and at a salt concentration corresponding to 1×SSC and 0.1% SDS.

5. A protein as defined in the following (C) or (D):
(C) A protein which comprises the amino acid sequence depicted in SEQ ID NO: 4; or
(D) A protein which comprises the amino acid sequence including deletion, substitution, insertion or addition of one or several amino acids in the amino acid sequence depicted in SEQ ID NO: 4 and which has at least one of the prephenate dehydratase activity or the chorismate mutase activity.

6. A DNA coding for a protein as defined in the following (C) or (D):
(C) A protein which comprises the amino acid sequence depicted in SEQ ID NO: 4; or
(D) A protein which comprises the amino acid sequence including deletion, substitution, insertion or addition of one or several amino acids in the amino acid sequence depicted in SEQ ID NO: 4 and which has at least one of the prephenate dehydratase or the chorismate mutase activity.

7. The DNA according to claim 6, which is a DNA as defined in the following (c) or (d):
(c) A DNA which comprises the nucleotide sequence depicted in SEQ ID NO: 3; or
(d) A DNA which is hybridizable with the nucleotide sequence depicted in SEQ ID NO: 3 or the probe prepared from said sequence under stringent condition and which code for a protein which has at least one of the prephenate dehydratase or chorismate mutase activity.

8. The DNA according to claim 7, wherein the stringent condition is the condition in which washing is performed at 60° C., and at a salt concentration corresponding to 1×SSC and 0.1% SDS.


SEQUENCE LISTING (4 sequences)

SEQ ID NO: 1 - DNA, 1083 bp, Methylophilus methylotrophus
  CDS (1)..(1080)
atgactgcatacgaaaaattagccaccgatgatgtgcgcgtgcttgaaatcaagccgctggtaaagcccgcggagctattgtctcgcctgcaggaaagtacagtcagtacccaaaacatccttaaaacgcggtcagcgattcatcatattctccatcagggcgacgaccggttgctggtgattgttggcccttgttccatccatgacacggaagctggcatggagtacgcgcgacgcctgctcgatgtgcgtcagcgactgggtggcgaattgctcattgtcatgcgcgtctattttgagaaaccccgtaccacggtagggtggaaaggcctgatcaacgacccgcatctggatgggacttatgatatcaatcttggattggagaaggcccgccgtttcctgctggatgtgaatgaaattggcatgcctgcagccacagaattcctcgatgtggtctccccgcaatatactgctgacctggtcagctggggagctattggcgctcggacgacagagtctcagattcaccgcgaattggcctctggcctgtcttgtccggttggctttaaaaatgggaccgatggcggcgtcaaagttgccattgatgcgattaaggcagcagccagtccgcatcactttttgtccgtgaccaaagaaggcgaatccgctatttttgccaccaagggtaatgaagactgccatgtgattttacgtggcggtaaagcgccgaactttgatgcgcctagtgtggcagcagtatgcgaccaattggcagacgctggcctggcaccggtattgatggtggattgcagtcatggcaatagccagaagcaatataaaaaccaaatttcggtggtgaatgatgtggctagccaaatagcgggtggagatgctcgcataatcgggatcatgctagagtcgcatttgaacgaagggcgacaggatcattcgccaggctgcagccttaattatgggcaatccatcaccgatgcctgtttgggatgggaggactcagtggctgtgctggaaacgctggctgctgcagtcaaggcccgccgtgacaagcatgccgctgctgaataa

SEQ ID NO: 2 - protein, 360 aa, Methylophilus methylotrophus
MTAYEKLATDDVRVLEIKPLVKPAELLSRLQESTVSTQNILKTRSAIHHILHQGDDRLLVIVGPCSIHDTEAGMEYARRLLDVRQRLGGELLIVMRVYFEKPRTTVGWKGLINDPHLDGTYDINLGLEKARRFLLDVNEIGMPAATEFLDVVSPQYTADLVSWGAIGARTTESQIHRELASGLSCPVGFKNGTDGGVKVAIDAIKAAASPHHFLSVTKEGESAIFATKGNEDCHVILRGGKAPNFDAPSVAAVCDQLADAGLAPVLMVDCSHGNSQKQYKNQISVVNDVASQIAGGDARIIGIMLESHLNEGRQDHSPGCSLNYGQSITDACLGWEDSVAVLETLAAAVKARRDKHAAAE

SEQ ID NO: 3 - DNA, 1083 bp, Methylophilus methylotrophus
  CDS (1)..(1080)
atgtctgatttattaaaacaatttagagataagattgacgcgattgatgcgcagattctagcgctcgtcaatgagcgtgccaagctggcacgtgaaatcggccatttaaaggatgatggtgtgatttaccgtcctgagcgtgaagcgcaaattatccgtcgcttgcaagcagaaaatgaagggccgctgtcaccggaggccgtcagccatattttccgtgcggtcatgtccaattgtcgcgctttggaaaaagaacttgcgattgcctttttgggcccactgggcacctacagtgaagaagccgcactcaagcagtttggtgaaggccgccaggcagtcgtctgcggcagtattgatgaagtttttcgtacggtggaagctggccaggcggattacggcgttgtccctgtagaaaactcaaccgaaggtgcggtgggaattacgctggacttattactgggtagtgcgctgcaagtggtaggcgaggtgactttaccagtacatcactgcttgctatcggcccagcaggatttgcaacagatcacgcatgtgttctcgcacgcacagtctttgtcgcaatgtcatgaatggctaaataaagtgttaccgagtgcacaacgagaagctgtgaccagcaacgcgcgtgctgcacaaatgattcatgagctagtcgccacccaaggcacgtttgcggctgcgattgccagcaaacgtgcggctgaattgtttgacttgaatatactcgccgaaaatatcgaagatgatccgaaaaataccacgcgctttctggtgttgggtaatcacggcgtcgcaccttctggtcaggataaaacctcgttggtgatgagtgctcacaacaagccaggcgcggtgttgcaattgctggaaccattgtcacgccatggcgtgagtatgaccaagctggaatcgcgtccatcacgtcaaaatctatggaactacgtattttttgttgacattgaaggtcatcaacagcagccctcggtacaagctgcgctgaaagaactggctgagcgcgcgactttccttaaagtgttgggctcatacccaaccgctattatttaa

SEQ ID NO: 4 - protein, 360 aa, Methylophilus methylotrophus
MSDLLKQFRDKIDAIDAQILALVNERAKLAREIGHLKDDGVIYRPEREAQIIRRLQAENEGPLSPEAVSHIFRAVMSNCRALEKELAIAFLGPLGTYSEEAALKQFGEGRQAVVCGSIDEVFRTVEAGQADYGVVPVENSTEGAVGITLDLLLGSALQVVGEVTLPVHHCLLSAQQDLQQITHVFSHAQSLSQCHEWLNKVLPSAQREAVTSNARAAQMIHELVATQGTFAAAIASKRAAELFDLNILAENIEDDPKNTTRFLVLGNHGVAPSGQDKTSLVMSAHNKPGAVLQLLEPLSRHGVSMTKLESRPSRQNLWNYVFFVDIEGHQQQPSVQAALKELAERATFLKVLGSYPTAII